(19) United States
(12) Patent Application Publication
(10) Pub. No.: US 2024/0335372 A1
(43) Pub. Date: Oct. 10, 2024

(54) Sustained-Release Vaginal Films

(71) Applicant: University of Pittsburgh-Of the Commonwealth System of Higher Education, Pittsburgh, PA (US)

(72) Inventors: Lisa Cencia Rohan, Pittsburgh, PA (US); Prithivirajan Durairajan, Pittsburgh, PA (US); Sravan Kumar Patel, Mars, PA (US)

(21) Appl. No.: 18/294,226

(22) PCT Filed: Aug. 2, 2022
(86) PCT No.: PCT/IB2022/057170

§ 371 Date: Feb. 1, 2024

Related U.S. Application Data
(60) Provisional application No. 63/228,387, filed on Aug. 2, 2021.

(51) Int. Cl. A61K 9/00; A61K 9/70; A61P 31/12

(52) U.S. Cl. CPC A61K 9/0036; A61K 9/7007; A61P 31/12

(57) ABSTRACT

Provided herein are stable, bioerodible films containing active ingredients, such as antimicrobial composition, antiviral compositions, antiretroviral compositions, and/or contraceptive compositions for intravaginal placement to provide prophylaxis against viral infections and contraception.

### CROSS-REFERENCE TO RELATED APPLICATIONS

The present application claims priority to U.S. Provisional Patent Application No. 63/228,387, filed Aug. 2, 2021, the disclosure of which is hereby incorporated by reference herein in its entirety.

### STATEMENT REGARDING FEDERALLY SPONSORED RESEARCH

This invention was made with government support under Grant Nos. AI142687 and AI120249 awarded by the National Institutes of Health, and GLOBALHEALTHBAA-M (VALUE Project within MATRIX) awarded by the USAID. The government has certain rights in the invention.

### BACKGROUND OF THE INVENTION

### Field of the Invention

The present invention relates to pharmaceutical vehicles for use in delivering therapeutic compositions to patients. More particularly, the present invention relates to films for sustained delivery of antiviral compositions to patients.

### Description of Related Art

Microbicides may be candidates applied vaginally or rectally for protection from sexually transmitted infections, including human immunodeficiency virus (HIV) infection. Preexposure prophylaxis (PrEP) with oral Truvada® (emtricitabine 200 mg/tenofovir disoproxil fumarate 300 mg)/emtricitabine reduces HIV acquisition in women who demonstrate high adherence to the daily medication. Likewise, when used consistently, vaginal dapivirine (DPV) ring and tenofovir (TFV) gels can confer protection. The ASPIRE and the Ring trials showed proportional increase in prevention efficacy with increased user adherence. One clinical trial of vaginal TFV gel demonstrated efficacy across the entire study population of sexually active women.

Even in trials of vaginal TFV gel which did not show a protective effect, sub-analyses suggest that for higher adherers, some protection was afforded. Understanding that adherence is central to PrEP effectiveness, developing a product that is easy to use and supports high adherence is an important objective. Significant efforts have been directed towards developing sustained delivery products such as injectables and vaginal rings which may improve adherence by reducing user error. However, these products are associated with various shortcomings, including fear of needles or expulsions after ring use. Accordingly, there is a need in the field for a vaginal drug delivery platform that permits on-demand, long-lasting delivery of drugs, such as microbicides and contraceptives, and that has minimal leakage, and can be discrete and portable.

### SUMMARY OF THE INVENTION

In view of the shortcomings in the field, vaginal film formulations providing sustained release of microbicides may provide an attractive alternative by delivering equivalent drug to the vagina while being more acceptable to women because of less leakage. Accordingly, provided herein is a bioerodible film for intravaginal delivery of an active agent, for example an antiretroviral composition, e.g. for treatment or prophylaxis of HIV.

Further non-limiting embodiments are set forth in the following numbered clauses:
1. A bioerodible film comprising one or more cellulose polymers, one or more non-cellulose polymers, and a therapeutic composition.
2. The bioerodible film according to clause 1, wherein the therapeutic composition is an antiviral composition.
3. The bioerodible film according to clause 1 or clause 2, wherein the antiviral composition is 4′-ethynyl-2-fluoro-2′-deoxyadenosine (EFdA) and/or a salt, prodrug, or derivative thereof.
4. The bioerodible film according to any of clauses 1-3, further comprising levonorgestrel (LNG).
5. The bioerodible film according to any of clauses 1-4, further comprising etonogestrel (ENG).
6. The bioerodible film according to any of clauses 1-5, further comprising levonorgestrel.
7. The bioerodible film according to any of clauses 1-6, further comprising etonogestrel.
8. The bioerodible film according to any of clauses 1-7, further comprising a mucoadhesive.
9. The bioerodible film according to any of clauses 1-8, further comprising a plasticizer.
10. The bioerodible film according to any of clauses 1-9, wherein the film comprises a first layer comprising hydroxypropyl methylcellulose, hydroxyethyl cellulose, hydroxypropyl cellulose, an acrylate-containing copolymer, polyethylene glycol, propylene glycol, EFdA, and LNG or ENG.
11. The bioerodible film according to any of clauses 1-10, wherein the film comprises a second layer comprising ethyl cellulose, glycerin, and LNG and/or ENG.
12. The bioerodible according to any of clauses 1-11, wherein the film comprises a second layer comprising poly (lactic-co-glycolic) acid.
13. The bioerodible film according to any of clauses 1-12, wherein the film comprises a plurality of layers.
14. The bioerodible film according to any of clauses 1-13, wherein the layers comprise: a first layer comprising hydroxypropyl methylcellulose, hydroxyethyl cellulose, hydroxypropyl cellulose, an acrylate-containing copolymer, polyethylene glycol, propylene glycol, EFdA, and LNG or ENG; a second layer arranged on the first layer and comprising ethyl cellulose, glycerin, and LNG or ENG; and a third layer arranged on the first layer and comprising ethyl cellulose and glycerin.
15. The bioerodible film according to any of clauses 1-14, wherein the layers comprise: a first layer comprising hydroxypropyl methylcellulose, hydroxyethyl cellulose, hydroxypropyl cellulose, an acrylate-containing copolymer, polyethylene glycol, propylene glycol, EFdA, and LNG; a second layer arranged on the first layer and comprising ethyl cellulose, glycerin, and LNG; and a third layer arranged on the first layer and comprising ethyl cellulose, glycerin, and LNG.
16. The bioerodible according to any of clauses 1-15, wherein the layers comprise: a first layer comprising hydroxypropyl methylcellulose, hydroxyethyl cellulose, hydroxypropyl cellulose, an acrylate-containing copolymer, polyethylene glycol, propylene glycol, EFdA, and LNG or ENG; a second layer arranged on the first layer and comprising poly (lactic-co-glycolic) acid; and a third layer arranged on the first layer and comprising poly (lactic-co-glycolic) acid.
17. The bioerodible film according to any of clauses 1-16, wherein the acrylate-containing copolymer comprises an ethyl acrylate, meth methacrylate, methacrylic acid ester copolymer.
18. The bioerodible film according to any of clauses 1-17, wherein the first layer comprises, by total weight of the layer: about 7% to about 15%, optionally about 11%. hydroxypropyl methylcellulose; about 4% to about 10%, optionally about 5%, optionally about 7%, optionally about 8%, hydroxyethyl cellulose; about 8% to about 20%, optionally about 10%, optionally about 14, optionally about 16%, hydroxypropyl cellulose; about 8% to about 20%, optionally about 11%, optionally about 16%, optionally about 18%, of acrylate-containing copolymer; about 6% to about 20%, optionally about 8%, optionally about 9%, optionally about 18%, polyethylene glycol; and about 4% to about 15%, optionally about 6%, optionally about 13%, propylene glycol.
19. The bioerodible film according to any of clauses 1-18, wherein the second layer comprises, by total weight of the layer: about 70% to about 80%, optionally about 71%, optionally about 77%, ethyl cellulose; and about 15% to about 25%, optionally about 18%, optionally about 19%, glycerin.
20. The bioerodible film according to any of clauses 1-19, wherein the third layer comprises, by total weight of the layer: about 60% to about 85%, optionally about 71%, optionally about 80%, ethyl cellulose; and about 15% to about 25%, optionally about 18%, optionally about 20%, glycerin.
21. The bioerodible film according to any of clauses 1-20, wherein the second and third layer comprise poly (lactic-co-glycolic) acid, optionally about 100%, by total weight of the layer, poly (lactic-co-glycolic) acid.
22. A method of delivering a therapeutic to a patient, comprising administering the bioerodible film according to any of clauses 1-21 to the patient.
23. The method according to clause 22, wherein the bioerodible film is inserted into the patient's vagina.
24. The method of clause 22 or clause 23, wherein the film comprises an antiviral composition and provides prophylactic levels of an antiviral composition for four weeks or more.
25. The method according to any of clauses 22-24, wherein the film comprises a contraceptive composition and prevents ovulation and/or fertilization for four weeks or more.

### BRIEF DESCRIPTION OF THE DRAWINGS

FIG. 1 is a schematic representation of how the films according to non-limiting embodiments described herein may be manufactured;

FIG. 2 shows the visual disintegration of biodegradable polymers incorporated at different concentrations in to the Eudragit polymer based film matrix over time. Intact films can be seen at time 0; The top row is 1% PCL, the following two rows are PLGA (1% and 5%), the next two rows are polylactide (1% and 5%), and the final two rows are PLACL (1% and 5%);

FIG. 3 shows the burst release of hydrophilic APIs (entecavir (ETV) and sofosbuvir (SBV)) and extended release of hydrophobic MK-2048 in the extended-release polymer matrix;

FIG. 4 is a schematic representation of how the films according to non-limiting embodiments described herein may be manufactured;

FIGS. 5A-5B show in vitro drug release comparisons for various films according to non-limiting embodiments described herein;

FIGS. 6A-6B show levels of EFdA (R-2.1, 2.2, and 2.3) and LNG (R-2.1 and 2.2) or ENG (R-2.3) in vaginal swabs obtained from Macaques;

FIGS. 7A-7B show EFdA (R-2.1, 2.2, and 2.3) and LNG (R-2.1 and 2.2) or ENG (R-2.3) levels in plasma obtained from Macaques;

FIGS. 8A-8B show EFdA and LNG levels in vaginal swabs obtained from Macaques;

FIGS. 9A-9B show EFdA and LNG levels in plasma obtained from Macaques;

FIGS. 10A-10B show percent release of EFdA and LNG in vitro in various non-limiting embodiments described herein; and

FIGS. 11A-11B show cross-sectional views of the R3.2 film, where the EFdA drug (green color) is loaded uniformly and only in the middle layer of the film.

### DESCRIPTION OF THE INVENTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed embodiments, whether provided in the examples or elsewhere herein.

The use of numerical values in the various ranges specified in this application, unless expressly indicated otherwise, are stated as approximations as though the minimum and maximum values within the stated ranges are both preceded by the word “about”. In this manner, slight variations above and below the stated ranges (e.g., ±10%, 9%, 8%, 7%, 6%, 5%, 4%, 3%, 2%, 1%, or 0.5%) can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “patient” or “subject” refers to members of the animal kingdom including but not limited to human beings and “mammal” refers to all mammals, including, but not limited to human beings.

As used herein, the “treatment” or “treating” of a wound or defect means administration to a patient by any suitable dosage regimen, procedure and/or administration route an amount of a composition, device or structure effective to, and with the object of achieving a desirable clinical/medical end-point, including attracting progenitor cells, healing a wound, correcting a defect, etc.

As used herein, the terms “comprising.” “comprise” or “comprised,” and variations thereof, are open ended and do not exclude the presence of other elements not identified. In contrast, the term “consisting of” and variations thereof is intended to be closed, and excludes additional elements in anything but trace amounts.

As used herein, a “polymer” is a compound formed by the covalent joining of smaller molecules, which are referred to herein as monomers before incorporation into the polymer and residues, or polymer subunits, after incorporated into a polymer. A “copolymer” is a polymer comprising two or more different residues. A polymer “comprises” or is “derived from” a stated monomer if that monomer is incorporated into the polymer. Thus, the incorporated monomer that the polymer comprises is not the same as the monomer prior to incorporation into a polymer, in that at the very least, certain terminal groups or atoms are incorporated into the polymer backbone or are excised. A polymer is said to comprise a specific type of linkage, such as an ester, or urethane linkage, if that linkage is present in the polymer.

Provided herein are stable pharmaceutical compositions (e.g. drug products) in the form of thin-film dosage forms of various therapeutic compositions that are advantageously delivered through mucosal tissue, for example vaginal mucosal tissue. The thin-films disclosed herein can be formulated to be bioerodible in a controlled fashion over time, thereby providing sustained release of therapeutic compositions. The thin films may be multi-layer films, with the same, or different, therapeutic compositions and/or polymer mixtures in each layer.

Therapeutic compositions/active ingredients useful in the thin films described herein can include, but are not limited to, antiretroviral compositions (e.g., nucleoside reverse transcriptase inhibitors, nonnucleoside reverse transcriptase inhibitors, protease inhibitors, fusion inhibitors, entry inhibitors, and integrase strand transfer inhibitors, such as, for example and without limitation, CSIC, efavirenz, emtricitabine, rilpivirine, atazanavir sulfate, darunavir ethanolate, elvitegravir, lamivudine, zidovudine, abacavir, zalcitabine, dideoxycytidine, azidothymidine, didanosine, dideoxyinosine, stavudine, rilpivirine, etravirine, delvaridine, nevirapine, amprenavir, tipranavir, inidinavir, saquinavir, lopinavir, ritonavir, fosamprenavir, ritonavir, darunavir, atazanavir, nelfinavir, enfuvirtide, raltegravir, dolutegravir, elvitegravir, maraviroc, DS003, tenofovir (TFV), TFV alefanamide, TFV disoproxil fumarate, dapivirine, and MK-2048), antiviral compositions (e.g., 4′-Ethynyl-2-fluoro-2′-deoxyadenosine (EFdA), nucleoside analogs, such as: acyclovir (2-amino-9-(2-hydroxyethoxymethyl)-3H-purin-6-one), penciclovir (2-amino-9-[4-hydroxy-3-(hydroxymethyl) butyl]-3H-purin-6-one), foscarnet (phosphonoformic acid), cidofovir ([(2S)-1-(4-amino-2-oxopyrimidin-1-yl)-3-hydroxypropan-2-yl]oxymethylphosphonic acid), adefovir (2-(6-aminopurin-9-yl) ethoxymethylphosphonic acid), and pharmaceutically-acceptable ester prodrugs thereof, such as valaciclovir (valine aciclovir ester, 2- [(2-amino-6-oxo-3H-purin-9-yl) methoxy] ethyl (2S)-2-amino-3-methylbutanoate) or famciclovir ([2-(acetyloxymethyl)-4-(2-aminopurin-9-yl) butyl] acetate)), antibiotic/antiprotozoal compositions (e.g., GRFT/Q-GRFT, metronidazole), antifungal compositions (e.g., clotrimazole). In addition, other compounds such as RANTES derivatives and retrocyclin (e.g., RC-101) can be included. Compositions that affect metabolism of another composition, such as antiretroviral compositions, such as cobicistat (sold under the trade name Tyboost®) can also be included. For example, a composition in a film as described herein can include atazanavir and cobicistat (sold under the trade name Evotaz®). In non-limiting embodiments, films according to the present invention include EFdA and/or EFdA-P, which have chemical structures as shown below:

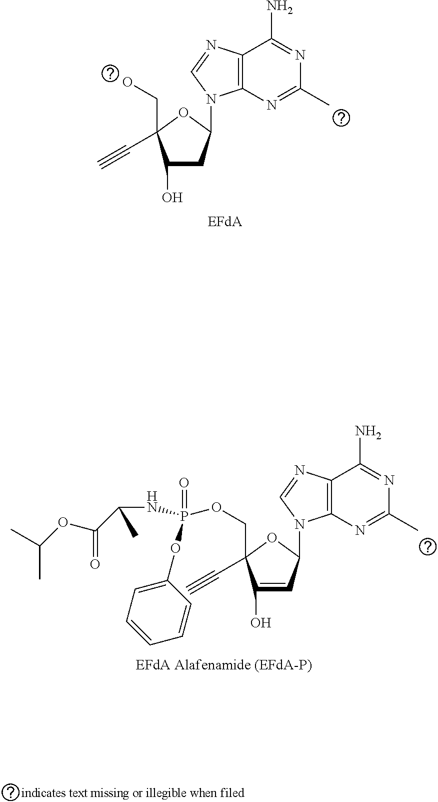 [SMILES: C#C[C@]1(CO)O[C@@H](n2cnc3c(N)nc(C)nc32)C[C@H]1O ; C#C[C@]1(CO[P@@](=O)(N[C@H](C)C(=O)OC(C)C)Oc2ccccc2)O[C@@H](n2cnc3c(N)nc(C)nc32)C=C1O]

Additional therapeutic compositions that may be included in the films disclosed herein include hormone and non-hormonal based contraceptive therapies, for example those utilized for birth control. Such compositions include natural and synthetic hormones and hormone receptor modulators, as well as any composition utilized for contraception, and will be referred to herein as “contraceptive compositions.” Such contraceptive compositions can include, without limitation, estrogens, progesterones, and/or progestins, including prodrugs and combinations thereof. In non-limiting embodiments, the composition includes an estrogen, a progesterone, a progestin, an estradiol (e.g., ethinyl estradiol), a norethindrine, a levonorgestrel (LNG), an etonogestrel (ENG), a desogrestrel, a dienogest, a hormonal receptor modulator (e.g., ulipristal acetate) a pro-drug of any of the foregoing, and combinations thereof, as well as like compositions known to those of skill in the art. In non-limiting embodiments, films according to the present invention include LNG and/or ENG.

Those of skill in the art will appreciate that the aforementioned examples are non-limiting, and that any therapeutic composition, including antiviral, antiretroviral, antibacterial, antiprotozoal, antifungal, or contraceptives, can advantageously be included in films described herein. In non-limiting embodiments, films according to the present invention include EFdA, EFdA-P, LNG, and/or ENG.

In a non-limiting embodiment, the film is a stable pharmaceutical composition in the form of a film for the intra-vaginal delivery of EFdA and a hormone (or pro-drug thereof), thus providing a combination of antiviral activity and contraception. In non-limiting embodiments, the film is formulated such that it provides sustained release of a microbiocide and/or a contraceptive composition for 1 week, 2 weeks, 3 weeks, 4 weeks, or longer, all values and subranges therebetween inclusive. In non-limiting embodiments, the film includes EFdA and one or both of LNG and ENG, and the film is configured (through the polymer mix) to release drugs for about 30 days.

The film disclosed herein includes one or more polymers that allow for a mechanically robust, stable, bioerodible matrix for the antiretroviral composition and/or contraceptive composition. In non-limiting embodiments, important physical characteristics of films according to the present invention include puncture strength, softness, and color. In non-limiting embodiments, the mixture of polymers includes one or more hydrophilic polymers, one or more hydrophobic polymers, one or more water-insoluble polymers, and one or more swelling polymers. In non-limiting embodiments, the film is formed from one or more cellulosic polymers, which are desirable for their biocompatible nature, including the lack of negative effect they, and their metabolites, have on innate lactobacilli flora and epithelium in the vagina. Those of skill will appreciate that for films described herein, toxicity to natural lactobacilli in vaginal tissue should be a minimum. Any cellulosic, e.g. cellulose-based, polymer may be useful in preparing films as described herein. In embodiments, the useful cellulosic polymers include hydroxypropyl cellulose (HPC), carboxymethylcellulose (NaCMC or CMC), hydroxypropyl methylcellulose (HMC or HPMC), ethyl cellulose (EC) and hydroxyethyl cellulose (HEC).

In non-limiting embodiments, the film includes one or more non-cellulose polymers. In some embodiments, the non-cellulose polymer is poly (lactide-co-glycolide) (PLG), poly (lactic-co-glycolic acid) (PLGA), polycaprolactone (PCL), polymethacrylate (PMA), polyvinyl pyrrolidone (PVP) or polyvinyl alcohol (PVA), polysaccharide polymers such as pullulan, polyacrylic acid polymers such as carbopol, and polymers including polyethylene glycol (PEG). In non-limiting embodiments, the film includes a copolymer of ethyl acrylate, methyl methacrylate and a low content of methacrylic acid ester with quaternary ammonium groups, available commercially under the tradename EUDRAGIT RS30D, EUDRAGIT RSPO, and/or EUDRAGIT NM 30D from Evonik Industries (Essen, Germany). In non-limiting embodiments, the film includes one or more hydrophilic polymers, one or more hydrophobic polymers, one or more water-insoluble polymers, and one or more swelling polymers.

To achieve a stable and bioerodible film, the various polymer components can be combined in a specific ratio or range for each component. As shown in the accompanying Examples, the amounts and/or ratios can be expressed in terms of a % w/w or by the weight of cach component. Further, those of skill in the art will appreciate that amounts and/or ratios present in a pre-film solution prior to casting, will differ from the amounts and/or ratios in the final film products. Converting amounts/ratios in a pre-film solution to the final film can be accomplished with a calculation as set forth below.

In addition to the polymer base of the film matrix, a film as described herein can include one or more additional components, for example, and without limitation, a plasticizer, a dispersant, a mucoadhesive, and/or a disintegrant. Suitable plasticizers for use in a film as described herein include glycerin, polyethylene glycol, polyethylene glycol monomethyl ether, propylene glycol, sorbitol sorbitan solution, diacetylated monoglycerides, castor oil, phthalates (such as bis (2-ethylhexyl) phthalate or dicthyl phthalate), triethyl citrate, tributyl citrate, trihexyl citrate, trioctyl citrate, diesters of dicarboxylic acids (such as sebacic acid or azelaic acid), and esters of glycerol (such as triacetin such as tributyrin). Suitable disintegrants include polyethylene glycol (PEG), such as PEG 400, PEG 6000, and PEG 8000. Suitable mucoadhesives include lectins, poly (ethylene glycol) (PEG), PVP, poly (acrylic acid) (PAA), poly (hydroxyethyl methacrylate) (PHEMA), HEC, HPC, HPMC, methylcellulose, NaCMC, thiolated polymers, carbophil, and chitosan.

In use, the polymers, active ingredient, and (if utilized) plasticizers, dispersants, mucoadhesives, and/or disintegrants may be admixed with any pharmaceutically acceptable carrier(s) or excipient(s) customarily used for administration of drugs to the patient in question (see, generally, Troy, DB, Editor, Remington: The Science and Practice of Pharmacy, 21st Ed., Lippincott Williams & Wilkins (2005), including pp. 871-888, 929-964, and 1018-1046, and 1626-1684 for non-limiting examples of various dosage forms, manufacturing methods, useful therapeutics, and useful analyses for designing a film for administration as described herein).

A film according to the present invention is utilized to deliver an amount effective of a therapeutic composition. An “amount effective” for treatment of a condition is an amount of an active agent or dosage form, effective to achieve a determinable end-point. The “amount effective” is preferably safe-at least to the extent the benefits of treatment outweighs the detriments and/or the detriments are acceptable to one of ordinary skill and/or to an appropriate regulatory agency, such as the U.S. Food and Drug Administration. Using the teachings of the present disclosure, a person of ordinary skill in the arts can prepare the film described herein, and titrate the effect on any objectively-determinable end-point, for instance first in an animal model and later in humans. As shown in the Examples below, an example of an “amount effective” is indicated. In embodiments, an “amount effective” is greater than or equal to 1 mg, 2 mg, 3 mg, 4 mg, 5 mg, 10 mg, 20 mg, 30 mg, or 40 mg, all values and subranges therebetween inclusive. In non-limiting embodiments, an “amount effective” of the microbicide composition is about 14 mg to about 30 mg (for example, based on sustained dwell and release, releasing about 1 mg/day for 30 days), e.g., about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, and/or about 30 mg, all values and subranges therebetween inclusive. In non-limiting embodiments, an “amount effective” of the contraceptive composition is about 1.5 mg (for example, based on sustained dwell and release, releasing about 50 μg/day for 30 days) to about 15 mg, e.g. about 1.5 mg, about 2 mg, about 3 mg, about 4, mg, about 5 mg, about 6 mg, about 7 mg, about 8 mg, about 9 mg, about 10 mg, about 11 mg, about 12 mg, about 13 mg, about 14 mg, and/or about 15 mg, all values and subranges therebetween inclusive. In non-limiting embodiments, an amount effective is about 14-16 mg of EFdA and 14-16 mg of LNG. Those of skill will appreciate that the amounts of the various therapeutic compositions may be adjusted for the target patient.

Those of skill in the art will appreciate that depending on the amount of therapeutic (e.g., antiviral and/or hormonal) composition to be delivered, the concentration in the film itself, and the dimensions of the film, will necessarily be adjusted accordingly; however, the selection of polymer will also affect the ultimate amount of active ingredient that can be used.

As described above, suitable films can include one or more layers. Each layer may include the same, or different, therapeutic compositions and/or polymer mixtures. Those of skill will appreciate that given the differences in structure and characteristics of many microbicides and contraceptive compositions, it may be necessary to include each in a distinct layer, with a distinct polymer mix that is suitable for each agent. In non-limiting embodiments, the film includes, 1, 2, 3, 4, 5, 6, 7, or more layers. In non-limiting embodiments, a film according to the present invention includes three layers, a middle layer including one or more microbicides, a bottom layer including one or more contraceptive compositions, and a top layer, optionally including no active ingredient. In non-limiting embodiments, a film according to the present invention includes three layers, a middle layer including one or more microbicides and one or more contraceptive compositions, and top and bottom layers, optionally including no active ingredient.

In non-limiting embodiments, a suitable film includes three layers, a first layer, a second layer arranged on a first surface of the first layer, and a third layer arranged on a second surface of the first layer, the second layer arranged opposite the third layer, such that, for example, the second and third layers sandwich the first layer. Within each layer, there may be any number of sublayers, for example a first (e.g., middle) layer may include 1, 2, 3, 4, or more sublayers, a second (e.g., top) layer may include 1, 2, 3, 4, or more sublayers, and a third layer (e.g. bottom) layer may include 1, 2, 3, 4, or more sublayers, each including the same or different polymers, active agents, and ratios of the components.

In non-limiting embodiments, the film has a first layer including hydroxypropyl methylcellulose, hydroxyethyl cellulose, hydroxypropyl cellulose, an acrylate-containing copolymer, polyethylene glycol, propylene glycol, EFdA, and LNG and/or ENG (optionally only LNG), a second layer arranged on the first layer and including ethyl cellulose glycerin, and LNG and/or ENG (optionally only LNG), and a third layer arranged on the first layer and including ethyl cellulose and glycerin.

In non-limiting embodiments, the film has a first layer including hydroxypropyl methylcellulose, hydroxyethyl cellulose, hydroxypropyl cellulose, an acrylate-containing copolymer, polyethylene glycol, propylene glycol, EFdA, and LNG and/or ENG (optionally only LNG), a second layer arranged on the first layer and including ethyl cellulose, glycerin, and LNG and/or ENG (optionally only LNG), and a third layer arranged on the first layer and comprising ethyl cellulose, glycerin, and LNG and/or ENG (optionally only LNG).

In non-limiting embodiments, the film has a first layer including hydroxypropyl methylcellulose, hydroxyethyl cellulose, hydroxypropyl cellulose, an acrylate-containing copolymer, polyethylene glycol, propylene glycol, EFdA, and LNG and/or ENG (optionally only LNG), a second layer arranged on the first layer and including poly (lactic-co-glycolic) acid, and a third layer arranged on the first layer and including poly (lactic-co-glycolic) acid.

In non-limiting embodiments, a first layer of a multi-layer film as described herein includes, by total weight of the layer, about 7% to about 15%, optionally about 11%, hydroxypropyl methylcellulose, about 4% to about 10%, optionally about 5%, optionally about 7%, optionally about 8%, hydroxyethyl cellulose, about 8% to about 20%, optionally about 10%, optionally about 14, optionally about 16%, hydroxypropyl cellulose, about 8% to about 20%, optionally about 11%, optionally about 16%, optionally about 18%, of an acrylate-containing copolymer, about 6% to about 20%, optionally about 8%, optionally about 9%, optionally about 18%, polyethylene glycol, and about 4% to about 15%, optionally about 6%, optionally about 13%, propylene glycol, all values and subranges therebetween inclusive.

In non-limiting embodiments, a second layer of a multi-layer film as described herein includes, by total weight of the layer, about 70% to about 80%, optionally about 71%, optionally about 77%, ethyl cellulose, and about 15% to about 25%, optionally about 18%, optionally about 19%, glycerin, all values and subranges therebetween inclusive.

In non-limiting embodiments, a third layer of a multi-layer film as described herein includes, by total weight of the layer, about 60% to about 85%, optionally about 71%, optionally about 80%, ethyl cellulose, and about 15% to about 25%, optionally about 18%, optionally about 20%, glycerin, all values and subranges therebetween inclusive.

In non-limiting embodiments, the second and third layer of a multi-layer film as described herein include, by total weight of the layer, about 100% poly (lactic-co-glycolic) acid.

In non-limiting embodiments, a first layer of a multi-layer film as described herein includes EFdA and LNG and/or ENG, optionally EFdA and LNG. In non-limiting embodiments, a second layer, and optionally a third layer, of a multi-layer film as described herein, include LNG and/or ENG, optionally LNG. In non-limiting embodiments, a second layer, and optionally a third layer, of a multi-layer film as described herein, include LNG, ENG, and/or EFdA, optionally LNG and EFdA.

In non-limiting embodiments, a first layer of a multi-layer film as described herein includes about 0.1% to about 3%, optionally about 0.4%., optionally about 1.2%, optionally about 2.4% of a contraceptive (e.g., LNG) and about 1% to about 4%, optionally about 1.8%, optionally about 3% of a microbicide (e.g., EFdA), all values and subranges therebetween inclusive. In non-limiting embodiments, a second and/or third layer includes about 0.1% to about 3%, optionally about 0.6%, optionally about 1.9%, of a contraceptive (e.g., LNG), all values and subranges therebetween inclusive.

As described below, one of skill can calculate the amount in a dried film layer from the above % w/w in the pre-film solution, and vice versa. In addition, for the foregoing, and for any amount disclosed herein, for a pre-film solution and/or a dried film, cach polymer, excipient (e.g., plasticizer, mucoadhesive, dispersant, and/or disintegrant) may be included in a listed amount ±25% and, for EC, Eudragit, and any polymers/components included in Eudragit, suitable amounts include the listed amount ±10%.

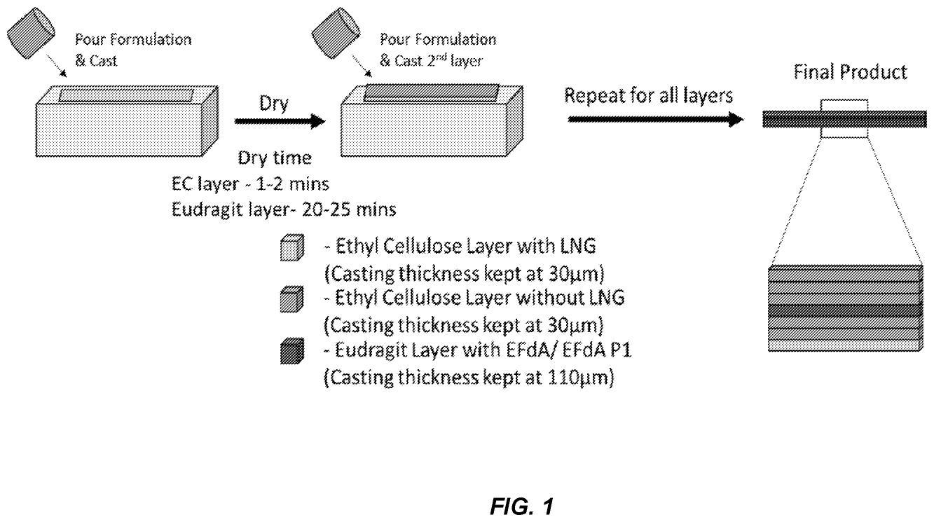

Also disclosed herein are methods of manufacturing a bioerodible film for intra-vaginal delivery of a microbicide composition and/or a contraceptive composition. Useful methods for producing a film as described herein include solvent casting. Solvent-casting techniques are known to those of skill in the art (see, e.g., Siemann, Solvent cast technology—a versatile tool for thin film production. Progr Colloid Polymer Sci 2005; 130:1-4). In that method, all ingredients are mixed in a solvent, for example purified water, in a specific addition order. The homogenous blend is then poured (cast) onto a heated or unheated surface and dried at a specific thickness to obtain thin polymeric film. An example process is shown in FIG. 1. Those of skill in the art will appreciate that the components included in the pre-film solution can affect the process steps. For example, casting EC is done with an unheated surface.

As noted above, the ratio of components included in the film imparts to the drug product mechanical robustness, stability, and dissolvability. Viscosity of the pre-film solution influences the formation of the film, thus, in embodiments, the polymer components are within a tight ratio window. In addition, the solvent casting process, including order of addition, process temperature and substrate type, are relevant.

A non-limiting protocol for forming a film as described herein includes dissolving/dispersing all the components in an appropriate solvent. Suitable solvents may be aqueous or organic in nature including, for example and without limitation, water, acetone, or ethanol. The order of component addition depends on the physical and chemical properties of the component, as well as the selected solvent. The active ingredient (e.g. antiretroviral) can be added to the polymer solution at various times dependent on the requirements of the composition. In many cases it is dissolved or suspended into the polymer-plasticizer. During mixing, entrapment of air in the solution can occur, and this can be eliminated using centrifugation or sonication for small batches or vacuumed for large scale production. Once the film solution is homogenous, it then cast onto a substrate and the film solution is dried. The drying process can occur at ambient temperature or at accelerated temperature by direct heat or using an oven. The drying process is defined for each specific formulation during film formulation development. Once the film sheets are prepared, they are cut into individual unit doses using a die press. The dimensions and shape of the film can be determined as needed, depending on the pharmacological application. Films can also be generated by directly applying the film solution into individual film molds.

A non-limiting protocol for producing a film as described herein includes, under constant mixing (e.g., at 350 RPM), adding accurately quantified amounts of a cellulose polymer, such as HEC, in a solvent, such as water, until dissolution. This step can be conducted under heating, for example about 85-90° C. Afterwards, further cellulose polymers, such as HPMC (e.g., HPMC K4M), may be added to the mixture. Following addition of HPMC, the temperature of the solution may be lowered, for example to about 65-70° C. Afterwards, further cellulose polymers, such as HPC (e.g., HPC JXF), may be added to the mixture. Afterwards, heating can be stopped, and the speed of mixing may be reduced (e.g., to about 200 RPM). Afterwards, further cellulose polymers (e.g., HPMC E5) may be added, for example when the temperature of the solution cools to about 30° C. Mixing may continue, for example at 100 RPM, for about 3-4 hours, for example until all excipients are dissolved and the solution is clear.

Thereafter, non-cellulose polymers may be added, for example PEG (e.g., PEG 400), and propylene glycol, as well as Eudragit (e.g., Eudragit RS30D), therapeutic composition(s), and any optional plasticizers. The solution may be mixed at 20 RPM for at least about 4-5 hours, optionally until a uniform mixture devoid of lumps is formed. Water loss may be calculated by weighing the mixture, and water may optionally be added.

In non-limiting embodiments, for example where multi-layer films are utilized, top and/or bottom layers including EC may be produced, by adding EX to a solvent (e.g., acetone) under mixing (e.g., 500-550 RPM). A plasticizer (e.g., glycerin) may then be added to the mixture, as may any therapeutic compositions. The mixture may be weighed to calculate solvent loss, and this lass may be compensated for. The mixture may be stored at 20 RPM for about 5-10 minutes.

In terms of casting the various layers, for a middle layer, a film applicator may be set to a heated temperature (e.g., about 72° C.). Vacuum may be applied, and films may be peeled off of the applicator surface after about 18-22 minutes. For bottom layers, the temperature of the applicator may be set to about 25° C., and films may be peeled off of the applicator after about 5 minutes. The middle layer film may then be placed on top of the bottom layer, for casting a top layer. Casting conditions for a top layer may be similar to, or the same as, the bottom layer. The multi-layer film may be air dried, for example for at least 30 minutes. Films may then be cut to any desirable size (e.g., 1″×1″).

In embodiments, a pre-film solution includes certain amounts of a component, and a final amount in the film product may be calculated as set forth below. Specifically, excipients in the formulation may be represented as percent weight by weight in a wet formulation. The total solid content of the formulation may be calculated by adding all the percent for the solids in the formulation recipe. For example, for a formulation with a total solid content of 23.07. To calculate the solid content of each excipient in a dried film (100 mg), the percent weight-by-weight of each excipient is divided by the total solid content of the formulation and multiplied by 100. Thus, for example, to calculate HPMC at a percent weight-by weight is 1.7, that value is divided by 23.07 and multiplied by 100 to get 7.37 mg. That is the solid content of HPMC in 100 mg of a dried film.

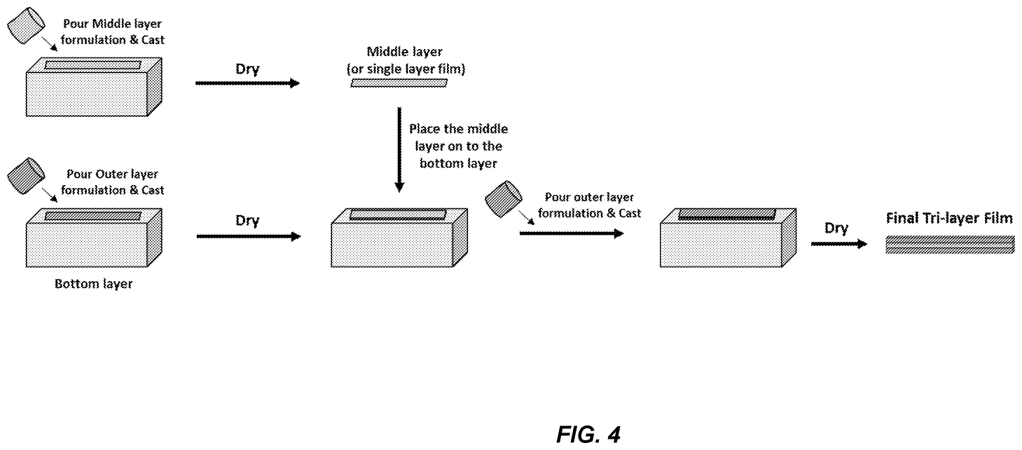

After the solution achieves a suitable pH, the solution is poured on an automatic thin film applicator for solvent casting. The applicator may be a heated surface. The solution is then dried. Suitable drying protocols can include a temperature of greater than 30° C., greater than 40° C., greater than 50° C., or greater than 60° C. Drying time will depend on the temperature used, but can be in the range of 5-35 minutes, all subranges therebetween inclusive. In one non-limiting embodiment, the solution is dried at about 71° C. for about 16 minutes. The produced film is then removed and cut to a suitable size, for example, 1 inch by 1 inch or 2 inches by 2 inches. As shown in FIGS. 1 and 4, in multi-layer films, multiple layers may be cast, one on another.

The film composition described herein bioerodes in vivo, and may be administered for a period of time, or at intervals, ranging from as needed to hourly, daily, weekly, monthly, or yearly, including increments therebetween, such as from one to six times per day, daily, every other day, weekly, bi-weekly, monthly, bi-monthly, quarterly, etc. An appropriate dosing schedule can be determined by a person of ordinary skill, such as a physician. In non-limiting embodiments, the films described herein bioerode and release active ingredient(s) such that replacement of the film in vivo may occur every 4 weeks or every 30 days.

Also provided herein is a method of delivering a therapeutic to a patient, where the therapeutic is provided in a bioerodible film as described herein. The method may include administering or inserting the film into an area of the patient, for example the vagina. The film may include any therapeutic composition(s) described herein. In non-limiting embodiments the film may include an antiviral composition, and may provide prophylaxis from a viral infection, such as HIV infection, for four weeks or more. In non-limiting embodiments, the film may include a contraceptive composition, and may prevent ovulation and/or fertilization for four weeks or more. Accordingly, methods of providing prophylactic treatment against a viral infection, such as an HIV infection, are within the scope of this disclosure, as are methods of preventing ovulation and/or fertilization.

### EXAMPLES

### Example 1

The goal of the work was to develop an extended release film platform incorporating an anti-HIV (EFdA-hydrophilic and EFdA P1-less hydrophilic) drug and a contraceptive drug (LNG or ENG-hydrophobic). The formulation development efforts are based from a previously developed sustained release formulation containing Eudragit.

An initial screening study was conducted to identify suitable polymers for this application. A panel of placebo films were manufactured incorporating Eudragit in addition with bio-degradable polymers (e.g. Polylactide, PLGA, poly (lactic acid-co-caprolactone) (PLACL) and PCL) as extended release modifying polymers. An exemplary formulation is shown in Table 1 below.

TABLE 1

| | | Solid Content |
|---|---|---|
| | | per 100 mg of |
| Polymers | Percent (w/w) | Film |
| Hydroxypropyl | 1.70% | 8.26 |
| methylcellulose E5 | | |
| Hydroxyethyl cellulose | 1.70% | 8.26 |
| Hydroxypropyl | 2.10% | 10.21 |
| methylcellulose K4M | | |
| Hydroxypropyl | 3.40% | 16.53 |
| cellulose JXF | | |
| Eudragit RS30D | 12.80% | 18.67 |
| PEG 400 | 1.82% | 8.85 |
| Propylene glycol | 1.32% | 6.42 |
| API (e.g. MK-2048) | 1.692% | 8.23 |
| MilliQ water | 73.5% | 14.58 |
| Total | 100.04% | 100.01 |

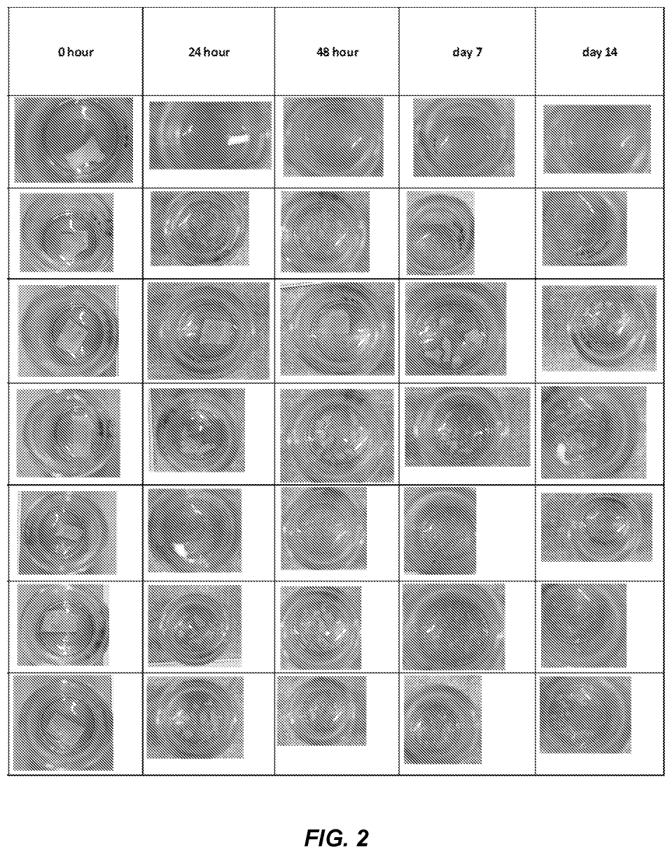

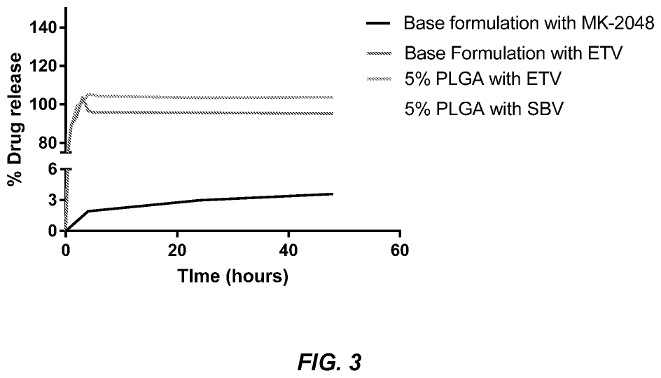

Based on visual disintegration it was identified that only the combination of PLGA and Eudragit polymers was successful in extending the disintegration time (FIG. 2). However, the incorporation of hydrophilic drugs (Entecavir (ETV)-EFdA surrogate or Sofosbuvir (SBV)-EFdA P1 surrogate) into this formulation showed burst release (within 60 minutes) (FIG. 3). This burst release could be attributed to the hydrophilic nature of the drug. This showed that the extended release polymer matrix showed sustained drug release for hydrophobic APIs while immediate release could be maintained with hydrophilic APIs.

In order to overcome burst release, the strategy employed was to add an outer ethyl cellulose layer that can act as a barrier and aid in slowing the diffusion of the drug. This strategy aided in the manufacture of film prototypes for Macaque studies.

### Example 2

The primary goal of the product development was to have sustained drug (EFdA/LNG) present in the vaginal space for 30 days. To achieve this goal, the strategy employed was to add an outer ethyl cellulose layer that can act as a barrier and aid in slowing the diffusion of the drug. Based on this hypothesis, the formulation for R 2.1 macaque study was developed. Table 2 below shows the formulation for the study as percent weight/weight and the solid content present in a 1″ by 1″ film. Briefly, HPMC K4M, and HPC polymers in the middle layer were incorporated for their film base, mucoadhesive, and sustained drug delivery properties. HPMC E5 and HEC were incorporated for their film base property. Eudragit NM30D is a non-degradable polymer which was used for its sustained drug release property. PEG 400 and propylene glycol was used as plasticizers for the middle layer film. The outer layer film includes ethyl cellulose (film base and sustained drug release) dissolved in acetone and triacetin as the plasticizer. LNG was loaded only in the outer most layer. The R 2.1 films contain 3 top and 3 bottom layers of ethyl cellulose based films and 1 middle layer of Eudragit NM30D based film. FIG. 1 shows the manufacturing process of the films.

TABLE 2

| | | | | R2.1 (solid content per | | |
|---|---|---|---|---|---|---|
| | | | | 100 mg of middle or outer | | |
| | R2.1 (% w/w) | | | layer dried film) (mg) | | |
| | | | Other | | | Other |
| | Middle | Bottom | outer | Middle | Bottom | outer |
| Polymers | Layer | layer | layers | Layer | layer | layers |
| Hydroxypropyl | 1.70% | — | — | 8.14 | — | — |
| methylcellulose E5 | | | | | | |
| Hydroxyethyl cellulose | 1.70% | — | — | 8.14 | — | — |
| Hydroxypropyl | 2.10% | — | — | 10.06 | — | — |
| methylcellulose K4M | | | | | | |
| Hydroxypropyl cellulose | 3.40% | — | — | 16.28 | — | — |
| JXF | | | | | | |
| Eudragit NM30D | 12.80% | — | — | 18.39 | — | — |
| PEG 400 | 1.82% | — | — | 8.72 | — | — |
| Propylene glycol | 1.32% | — | — | 6.32 | — | — |
| Ethyl cellulose | — | 12.00% | 12.00% | — | 76.92 | 80 |
| Triacetin | — | 3.00% | 3.00% | — | 19.23 | 20 |
| Acetone | — | 84.40% | 85.00% | — | — | — |
| LNG | — | 0.6% | — | — | 3.85 | — |
| EFdA/EFdA P1 | 2% | — | — | 9.58 | — | — |
| MilliQ water | 73.16% | — | — | 14.37 | — | — |
| Total | 100.00% | 100.00% | 100.00% | 100.00 | 100.00 | 100.00 |

The weight of the films (1″ by 1″) were measured using Mettler Toledo (Model # MS204TS) analytical instrument. The thickness of the films was measured using Mitutoyo (Model #547-520) thickness gauge instrument.

The water content of the film was measured using Metrohm Karl Fischer oven titration method. Briefly, the sample was heated at 150° C. and the evaporated water is titrated against iodine solution to calculate the percentage of water present in the solution.

The puncture strength of the film was evaluated to measure the tensile property of the film. The puncture strength of the film was tested using Texture Analyzer instrument. The films to be tested were placed inside the TA 1085-5 platform and TA8A 1/8′ probe will puncture the film at a constant test speed of 3 mm/s. The texture analyzer will measure the maximum force which is required to puncture the film and then it will be normalized to the thickness of each film tested. The value in the table represents the average and standard deviation of puncture strength (kg/mm).

The disintegration time of the film was tested in-vitro to evaluate which formulation will have a better disintegration profile and thereby can sustain the release of drug. The disintegration time of the films were tested using Texture Analyzer instrument. The films to be tested were placed inside the TA 1085-5 platform and TA8A 1/8′ probe will puncture the film at a constant test speed of 0.2 mm/s after 15 μL of MilliQ water is placed on the interface of probe and film. The texture analyzer will measure the time which is required to disintegrate the film and then it will be normalized to the thickness of each film tested. The table represents the average and standard deviation of disintegration time (s/mm).

Contact angle measurement was performed to evaluate the surface hydrophobic nature of the films. The contact angle of the film was tested using Biolin Scientific One Attension contact angle measurement instrument. Briefly, 8 μL of MilliQ water was placed on the film and the contact angle was recorded over time. Table 3 below shows the average contact angle (°) recorded, 5 seconds after the drop was placed on the film.

The drug content of the film was analyzed to find the average drug loading per film as compared to the label claim. Briefly, 1″ by 1″ films were placed in 50 ml volumetric flask and stirred for one day using 70% MeOH as a solvent. Extract samples were analyzed using reverse phase HPLC and drug amount in sample extract was obtained from a calibration curve regression line.

In vitro drug dissolution was performed to evaluate the drug release profile of the formulations developed. To perform the drug dissolution a 1″ by 1″ film was used in a Distek dissolution apparatus. 12 ml of Vaginal Fluid Simulant (VFS) was used as the dissolution media and 1 ml of media was sampled out and replaced. The percent drug release was calculated and plotted for each of the formulation.

TABLE 3

| | R2.1 EFdA/LNG | | R2.1 EFdA P1/LNG | |
|---|---|---|---|---|
| Physical Characterization | Average | SD | Average | SD |
| Weight (mg) | 252.90 | 43.70 | 207.90 | 11.20 |
| Thickness (mm) | 0.98 | 0.24 | 0.70 | 0.12 |
| Puncture Strength (Kg/mm) | 1.50 | 0.46 | 1.70 | 0.40 |
| Disintegration Time (s/mm) | 8093.80 | 1498.90 | 10615.90 | 679.00 |
| Water Content (%) | 1.33 | 0.08 | 2.41 | 0.19 |
| Drug content (mg) | 11.71 | 3.83 | 9.88 | 0.36 |
| (EFdA/EFdA P1) | | | | |
| Drug content (mg) (LNG) | 0.88 | 0.21 | 1.14 | 0.10 |

These films showed sustained drug release in vitro and therefore were evaluated for pharmacokinetics in the macaque model. Platform was further optimized with respect to number of layers, drug amounts and processing conditions in the following Examples.

### Example 3

To further optimize film appearance and tactile properties, R2.2 the 2.3 films were developed. Tables 4 and 5 below show the formulation developed for the study as percent weight/weight and the solid content present in a 1″ by 1″ film, and physical characteristics of the films. Briefly, the number of outer layers were reduced from 3 to 1 for each top and bottom layer. Also, triacetin was replaced with glycerin as plasticizer for the outer layer. The Eudragit NM30D was replaced with Eudragit RS30D in the middle layer formulation. LNG was added to the middle layer as well. The manufacturing process also differed as the middle layer was manufactured separately and coated with the outer layers. FIG. 4 shows the manufacturing process of the films.

TABLE 4

| | | | | R2.2 (solid content per | | |
|---|---|---|---|---|---|---|
| | R2.2 | | | 100 mg of middle or outer | | |
| | R2.2 (% w/w) | | | layer dried film) (mg) | | |
| | Middle | Bottom | Top | Middle | Bottom | Top |
| Polymers | Layer | layer | layer | Layer | layer | layer |
| Hydroxypropyl | 1.70% | — | — | 8.08 | | |
| methylcellulose | | | | | | |
| E5 | | | | | | |
| Hydroxyethyl | 1.70% | — | — | 8.08 | | |
| cellulose | | | | | | |
| Hydroxypropyl | 2.10% | — | — | 9.99 | | |
| methylcellulose | | | | | | |
| K4M | | | | | | |
| Hydroxypropyl | 3.40% | — | — | 16.17 | | |
| cellulose JXF | | | | | | |
| Eudragit RS30D | 12.80% | — | — | 18.26 | | |
| PEG 400 | 1.82% | — | — | 8.65 | | |
| Propylene glycol | 1.32% | — | — | 6.28 | | |
| Ethyl cellulose | — | 12.00% | 12.00% | — | 76.92 | 80.00 |
| Glycerin | — | 3.00% | 3.00% | — | 19.23 | 20.00 |
| Acetone | — | 84.40% | 85.00% | — | — | — |
| API (LNG) | 0.35% | 0.60% | | 1.66 | 3.85 | |
| API (EFdA) | 1.8% | — | — | 8.56 | | |
| MilliQ water | 73.01% | — | — | 14.27 | | |
| Total (g) | 100.00% | 100.00% | 100.00% | 100 | 100.00 | 100.00 |

TABLE 5

| | R2.2 EFdA/LNG | | R2.2 EFdA P1/LNG | |
|---|---|---|---|---|
| Physical Characterization | Average | SD | Average | SD |
| Weight (mg) | 173.45 | 8.46 | 186.05 | 7.80 |
| Thickness (mm) | 0.43 | 0.03 | 0.36 | 0.02 |
| Puncture Strength (Kg/mm) | 1.10 | 0.16 | 3.00 | 0.68 |
| Disintegration Time (s/mm) | 50.14 | 1.19 | 1169.07 | 680.05 |
| Water Content (%) | 1.78 | 0.00 | 1.75 | 0.00 |
| Drug content (mg) | 8.89 | 0.39 | 8.62 | 0.59 |
| (EFdA/EFdA P1) | | | | |
| Drug content (mg) (LNG) | 3.34 | 0.13 | 3.39 | 0.08 |

Note that the disintegration time method was altered by changing the force from 10 g (used for R2.1) to 200 g (used for R2.2, 2.3, 3.1, and 3.2) and the amount of water added was 60 μL instead of 15 μL.

R-2.2 films were also evaluated in the macaque study. Films showed sustained release for both drugs in macaque study.

### Example 4

Studies to validate utilization of different hormonal actives were carried out. R-2.3 films were similar to R-2.2 films except the film was made with EFdA and ENG combo instead of EFdA and LNG combo. Tables 6 and 7 below show the formulation and physical characteristics.

TABLE 6

| | R2.3 | | | | | |
|---|---|---|---|---|---|---|
| | | | | R2.3 (solid content per | | |
| | | | | 100 mg of middle or outer | | |
| | R2.3 (% w/w) | | | layer dried film) (mg) | | |
| | Middle | Bottom | Top | Middle | Bottom | Top |
| Polymers | Layer | layer | layer | Layer | layer | layer |
| Hydroxypropyl | 1.70% | — | — | 8.08 | | |
| methylcellulose E5 | | | | | | |
| Hydroxyethyl | 1.70% | — | — | 8.08 | | |
| cellulose | | | | | | |
| Hydroxypropyl | 2.10% | — | — | 9.99 | | |
| methylcellulose K4M | | | | | | |
| Hydroxypropyl | 3.40% | — | — | 16.17 | | |
| cellulose JXF | | | | | | |
| Eudragit RS30D | 12.80% | — | — | 18.26 | | |
| PEG 400 | 1.82% | — | — | 8.65 | | |
| Propylene glycol | 1.32% | — | — | 6.28 | | |
| Ethyl cellulose | — | 12.00% | 12.00% | — | 76.92 | 80.00 |
| Glycerin | — | 3.00% | 3.00% | — | 19.23 | 20.00 |
| Acetone | — | 84.40% | 85.00% | — | — | — |
| API (ENG) | 0.35% | 0.60% | | 1.66 | 3.85 | |
| API (EFdA) | 1.8% | — | — | 8.56 | | |
| MilliQ water | 73.01% | — | — | 14.27 | | |
| Total (g) | 100.00% | 100.00% | 100.00% | 100 | 100.00 | 100.00 |

TABLE 7

R2.3 EFdA/ENG

| Physical Characterization | Average | SD |
|---|---|---|
| Weight (mg) | 183.25 | 8.86 |
| Thickness (mm) | 0.29 | 0.02 |
| Puncture Strength (Kg/mm) | 0.85 | 0.19 |
| Disintegration Time (s/mm) | 548.70 | 651.41 |
| Water Content (%) | 3.75 | 0.14 |
| Drug content (mg) (EFdA) | 8.43 | 0.27 |
| Drug content (mg) (ENG) | 3.76 | 0.20 |

The films developed were similar to R-2.2 films except that the combination of drug chosen was EFdA/ENG to evaluate in the Macaque studies.

### Example 5

The R-3.1 films are similar to R-2.2 films except the film was made with increased EFdA and LNG drug loading and LNG was added to all three layers (middle and outer layers). The formulation is shown in Table 8 below.

TABLE 8

| | | | R3.1 (solid content per 100 mg of | |
|---|---|---|---|---|
| | | | middle or outer layer dried film) | |
| | R3.1 (% w/w) | | (mg) | |
| | Middle | Top and | Middle | Top and |
| Polymers | Layer | bottom layers | Layer | bottom layer |
| Hydroxypropyl | 1.70% | — | 7.37 | — |
| methylcellulose E5 | | | | |
| Hydroxyethyl | 1.70% | — | 7.37 | — |
| cellulose | | | | |
| Hydroxypropyl | 2.10% | — | 9.10 | — |
| methylcellulose K4M | | | | |
| Hydroxypropyl | 3.40% | — | 14.74 | — |
| cellulose JXF | | | | |
| Eudragit RS30D | 12.80% | — | 16.64 | — |
| PEG 400 | 1.82% | — | 7.89 | — |
| Propylene glycol | 1.32% | — | 5.72 | — |
| Ethyl cellulose | — | 12.00% | — | 70.92 |
| Glycerin | — | 3.00% | — | 17.73 |
| Acetone | — | 83.08% | — | — |
| LNG | 1.22% | 1.92% | 5.29 | 11.35 |
| EFdA | 2.97% | — | 12.87 | — |
| MilliQ water | 70.97% | — | 13.00 | — |
| Total (g) | 100.00% | 100.00% | 100.00 | 100.00 |

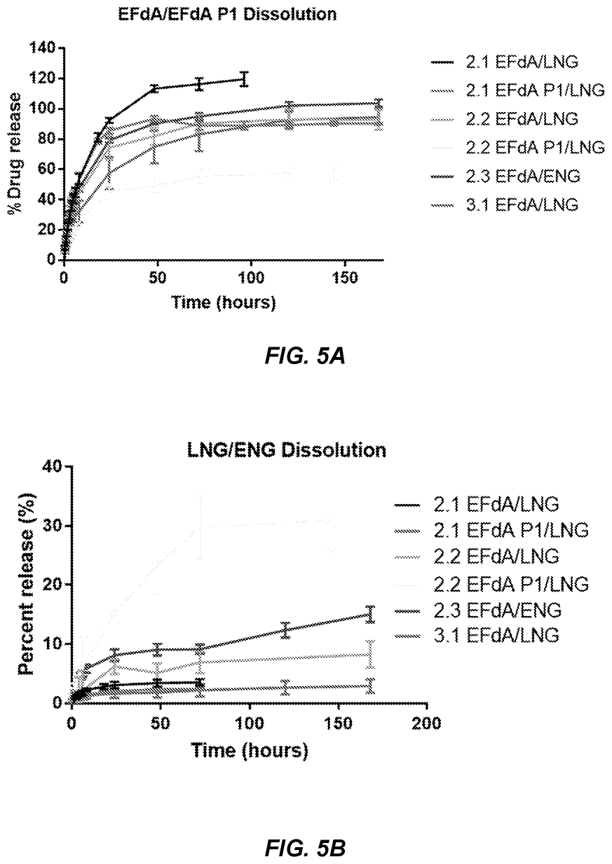

The films developed were similar to R-2.2 films but differed in drug loading and distribution of the drug among the three layers of films. These studies demonstrate the versatility of the platform to accommodate drug within different layers and achieve varied release patterns. Dissolution is shown in FIGS. 5A-5B.

Table 9 below summarizes the characteristics of the films formed as described above, and Table 9 below summarizes the physiochemical properties of the various therapeutic compositions included in the films.

TABLE 9

| | | | | | | Study 3 | Study 4 |
|---|---|---|---|---|---|---|---|
| | | Study 1 (R 2.1) | | Study 2 (R 2.2) | | (R 2.3) | (R 3.1) |
| | | EFdA/ | EFdA | EFdA/ | EFdA | EFdA/ | EFdA/ |
| | | LNG | P1/LNG | LNG | P1/LNG | ENG | LNG |
| Characterization | | Average ± | Average ± | Average ± | Average ± | Average ± | Average ± |
| (1″ × 1″ film) | | SD | SD | SD | SD | SD | SD |
| Weight (mg) | | 252.90 ± | 207.90 ± | 173.45 ± | 186.05 ± | 183.25 ± | 199.46 ± |
| | | 43.7 | 11.2 | 8.46 | 7.8 | 8.86 | 10.51 |
| Thickness (mm) | | 0.98 ± | 0.70 ± | 0.43 ± | 0.36 ± | 0.29 ± | 0.331 ± |
| | | 0.24 | 0.12 | 0.03 | 0.02 | 0.02 | 0.02 |
| Drug content | | 11.71 ± | 9.88 ± | 8.89 ± | 8.62 ± | 8.43 ± | 15.32 ± |
| EFdA/EFdA P1 | | 3.83 | 0.36 | 0.39 | 0.59 | 0.28 | 1.77 |
| (mg) | | | | | | | |
| Drug content | | 0.88 ± | 1.14 ± | 3.34 ± | 3.39 ± | 3.76 ± | 16.61 ± |
| LNG/ENG (mg) | | 0.21 | 0.1 | 0.13 | 0.08 | 0.2 | 1.39 |
| Release | ARV | 1.4 | 1.6 | 0.9 | 0.5 | 0.9 | 1.4 |
| rate (mg/√h) | Progestin | 0.005 | 0.004 | 0.03 | 0.1 | 0.05 | 0.05 |

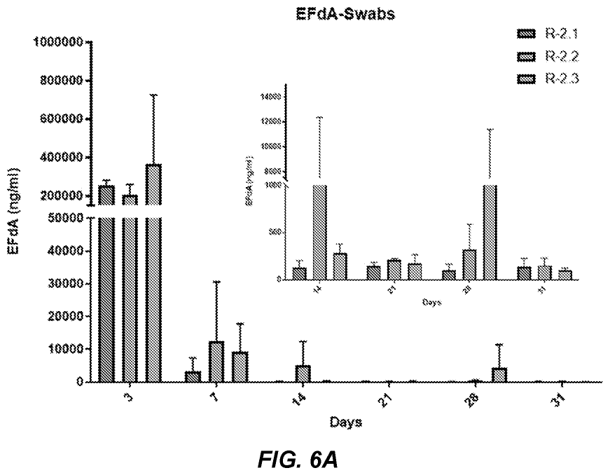

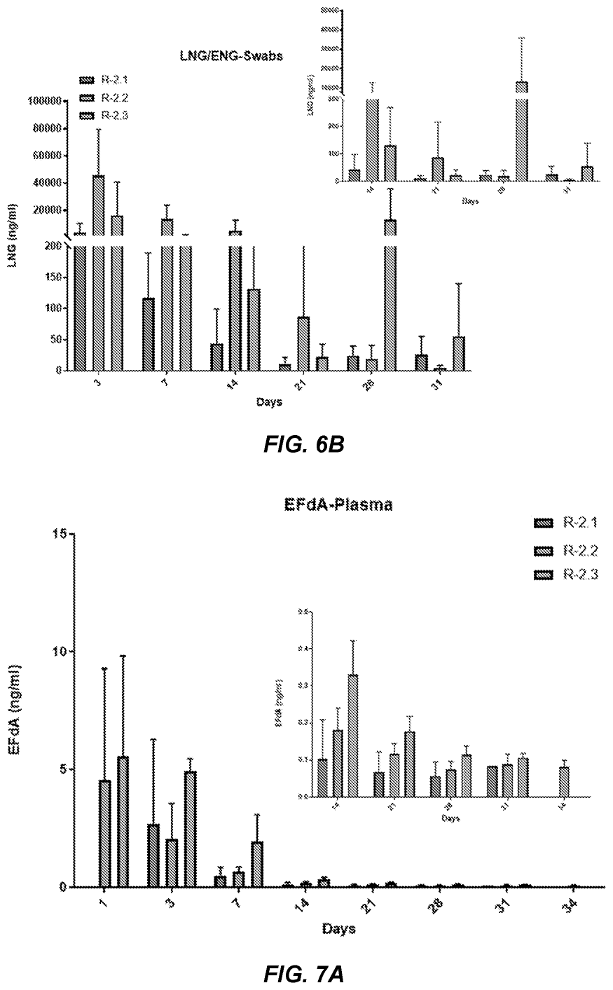

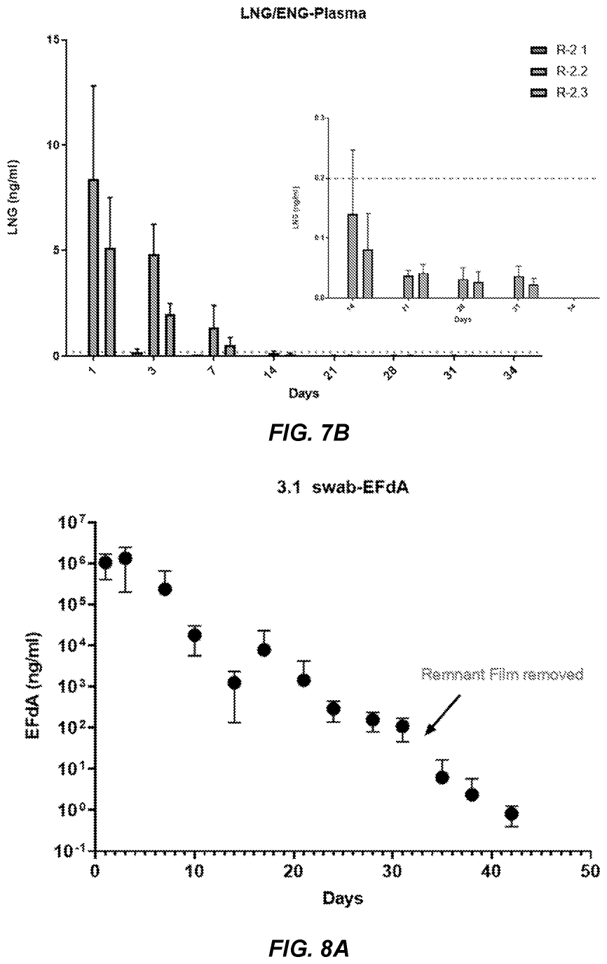

EFdA was detected until day 31 in swabs for all animals and for LNG swabs levels 2/3 animals from D7 onwards were above BLQ (R-2.1). Results are shown in FIGS. 6A-7B.

As can be appreciated from the FIGS. 6A-7B, 2/3 animals on D28 and 1/3 on D31 were above BLQ (R-2.1); LNG: 1/3 animals on D7 were above BLQ and 0/3 from D14 onwards (R-2.1).

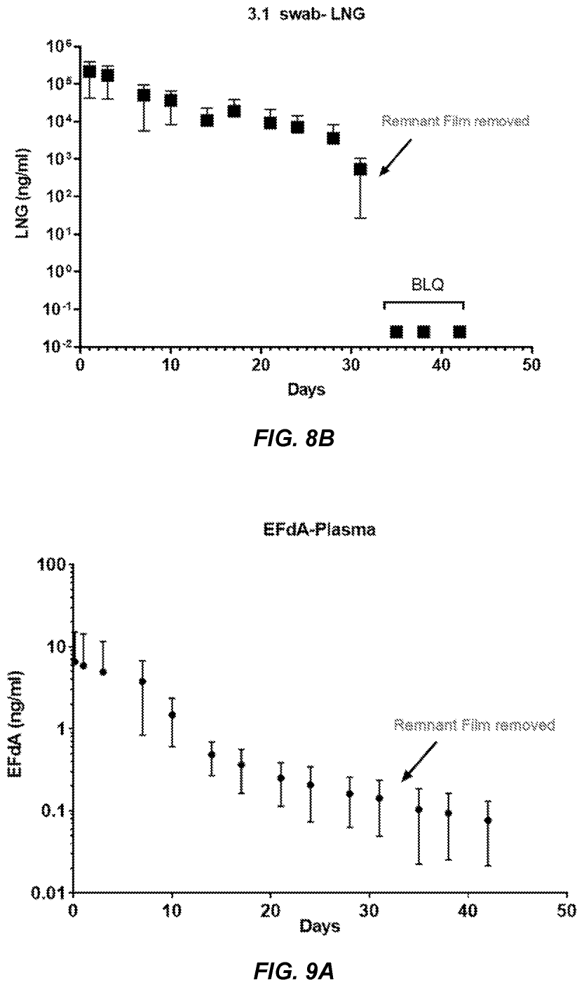

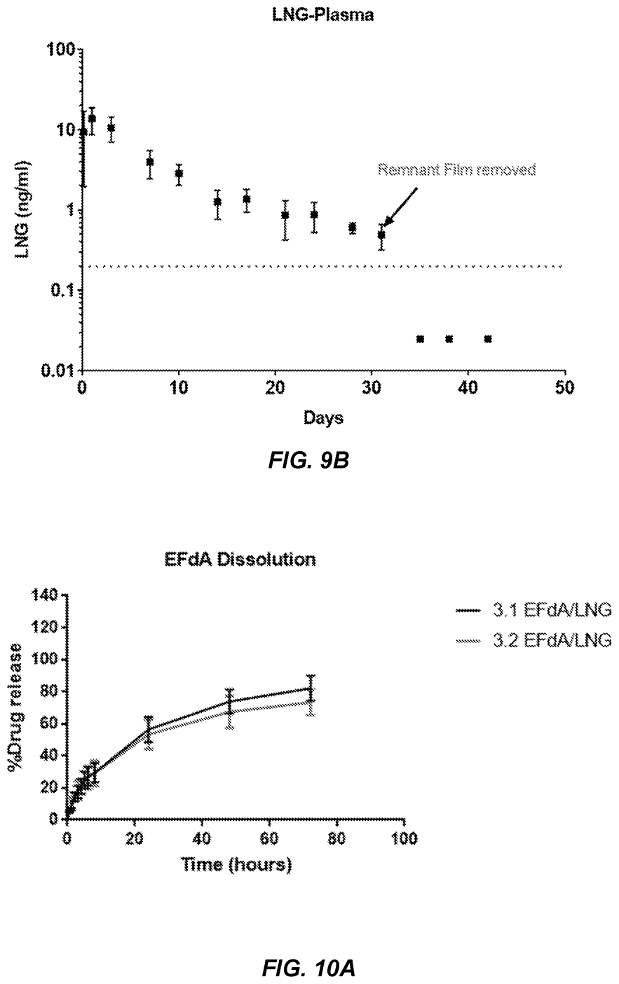

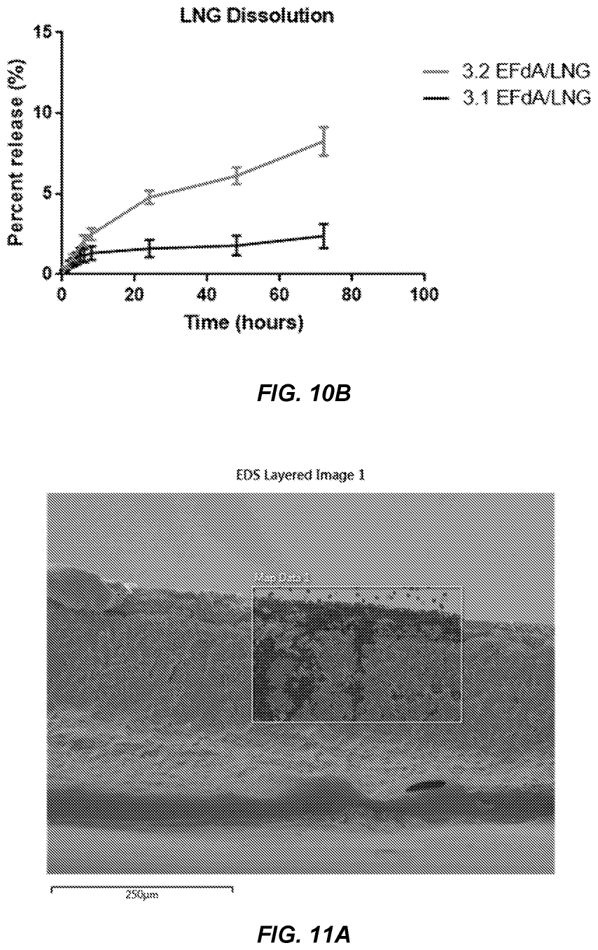

From FIGS. 8A-9B, it can be seen that EFdA and LNG release for 30 days was achieved for both vaginal swab and plasma levels in the macaque.

The NHP studies demonstrate utility of the film for sustained vaginal administration in an in vivo model. In NHP film evaluations, both vaginal fluid and blood components (e.g. plasma) samples were collected for at least 31 days post-film administration to assess drug levels and evaluate vaginal release and systemic exposure. The first prototype multilayer ER MPT films containing EFdA/LNG or EFdA-P1/LNG confirm that, on average, detectable levels of EFdA (or EFdA-P1) and LNG were found to be present in the vaginal fluid until day 31 for both films in most animals, but not all. In study 2, MPT films with increased LNG dose (e.g., 3.5 mg vs. 1 mg) were evaluated. For EFdA/LNG film, detectable vaginal and plasma EFdA and LNG levels were observed until day 31 in all animals. Study 3 utilized the same formulation developed for Study 2 by simply replacing LNG with ENG. Results showed that this film also achieved sustained drug levels in vivo for ENG and EFdA.

MPT film was further optimized to accommodate increased dose levels of LNG (16 mg) and EFdA (14 mg). The developed MPT film was administered in six macaques. Detectable EFdA and LNG swab levels were obtained in vaginal fluids supporting successful drug delivery for 1-month. Plasma concentrations showed immediate uptake of EFdA and LNG at the first evaluated time point e.g., 3-4 hours after administration (FIGS. 9A-9B). The plasma half-life for EFdA and LNG were found to be significantly higher than previously reported values after oral administration, supporting the sustained release property of the ER film platform. The plasma Cₘₐₓ for EFdA and LNG were found to be 8.66 and 15.5 ng/ml. Importantly, efficacious levels of LNG were obtained in plasma for the entire duration of the study (FIGS. 9A-9B). The mean EFdA-TP levels in PBMCs were found to range 0.36-0.018 pmol/10⁶ cells from day 1-31. Levels above the target (0.05 pmol/10⁶ PBMCs) were found until at least day 14, and remained detectable at an average concentration of 0.018 pmol until day 31. The EFdA-TP levels confirm extended protection from MPT film for a minimum duration of 1-month after accounting for 4-5 times faster elimination half-life for macaques (˜40 h) compared to humans (190h). Overall, these results clearly suggest that the developed MPT film could provide both immediate and sustained 1-month protection from HIV infection and unintended pregnancy after film administration, and additionally offers extended window of protection for HIV prevention.

### Example 6

Based on the foregoing, a non-limiting embodiment of a suitable formulation for sustained release of therapeutic compositions in vivo may include components in the amounts set forth in Table 10 below, with physical characteristics as set forth in Table 11.

TABLE 10

| | | | R3.2 (solid content per 100 mg of | |
|---|---|---|---|---|
| | | | middle or outer layer dried film) | |
| | R3.2 (% w/w) | | (mg) | |
| | Middle | Top and | Middle | Top and |
| Polymers | Layer | bottom layers | Layer | bottom layers |
| Hydroxypropyl | 1.70% | — | 5.41 | — |
| methylcellulose E5 | | | | |
| Hydroxyethyl | 1.70% | — | 5.41 | — |
| cellulose | | | | |
| Hydroxypropyl | 2.10% | — | 6.69 | — |
| methylcellulose K4M | | | | |
| Hydroxypropyl | 3.40% | — | 10.82 | — |
| cellulose JXF | | | | |
| Eudragit RS30D | 12.80% | — | 12.23 | — |
| PEG 400 | 5.93% | — | 18.88 | — |
| Propylene glycol | 4.33% | — | 13.79 | — |
| PLGA | — | 5.00% | — | 100 |
| Acetone | — | 95.00% | — | — |
| LNG | 2.44% | — | 7.77 | — |
| EFdA | 2.97% | — | 9.46 | — |
| MilliQ water | 62.63% | — | 9.55 | — |
| Total (g) | 100.00% | 100.00% | 100.00 | 100.00 |

TABLE 11

| Characterization | R3.2 |
|---|---|
| Weight (mg) | 160 ± 9.7 |
| Thickness (mm) | 0.26 ± 0.03 |
| #Folds Till break (N = 3) | 150+ |
| Puncture Strength (Kg/mm) | 1.88 ± 0.39 |
| Drug content (mg) | 16.82 ± 0.62 (EFdA) |
| | 14.71 ± 0.74 (LNG) |
| Water Content (%) | 4 ± 0.1 |

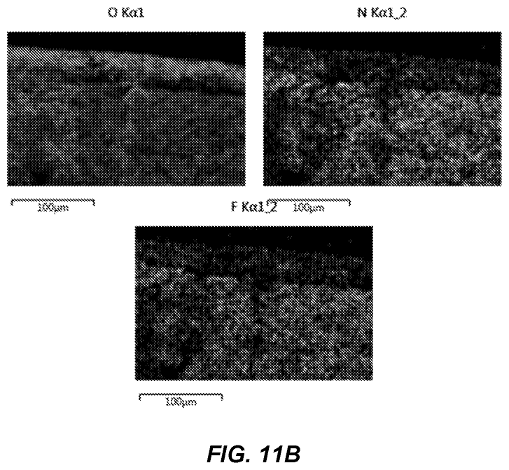

The films developed were similar 3.1 films but differed in distribution of the drug among the three layers of films, the amount of plasticizer and the outer layer coating. The films were soft, flexible and opaque in comparison to the 3.1 films even with increased drug loading. FIGS. 11A-11B show cross-sectional views of the R3.2 film, where the EFdA drug (green color) is loaded uniformly and only in the middle layer of the film.

In conclusion, this data demonstrates the ability for the single and multilayer formulations to achieve desired drug release profiles, which included both immediate and sustained release for single and combination drugs of both hydrophilic and hydrophobic nature.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A bioerodible film comprising one or more cellulose polymers, one or more non-cellulose polymers, and a therapeutic composition.

2. The bioerodible film according to claim 1, wherein the therapeutic composition is an antiviral composition.

3. The bioerodible film according to claim 1, wherein the antiviral composition is dapavirine and/or a salt, prodrug, or derivative thereof.

4. The bioerodible film according to claim 2, further comprising levonorgestrel (LNG) and/ore estel (ENG).

5. (canceled)

6. The bioerodible film according to claim 3, further comprising LNG and/or ENO.

7. (canceled)

8. The bioerodible film according to claim 1, further comprising a mucoadhesive and/or a plasticizer.

9. (canceled)

10. The bioerodible film according to claim 1, wherein the film comprises a first layer comprising hydroxypropyl methylcellulose, hydroxyethyl cellulose, hydroxypropyl cellulose, an acrylate-containing copolymer, polyethylene glycol, propylene glycol, 4′-ethyny-2-fluoro-2′-deoxyadenosine (EFdA) and/or a salt, prodrug, or derivative thereof, and LNG or ENG.

11. The bioerodible film according to claim 10, wherein the film comprises a second layer comprising:
ethyl cellulose, a plasticizer, and LNG, and/or ENG; and/or
poly(lactic-co-glycolic) acid.

12. (canceled)

13. (canceled)

14. The bioerodible film according to claim 1, wherein the film comprises a plurality of layers, wherein the layers comprise:
a first layer comprising hydroxypropyl methylcellulose, hydroxyethyl cellulose, hydroxypropyl cellulose, an acrylate-containing copolymer, polyethylene glycol, propylene glycol, EFdA and/or a salt, prodrug, or derivative thereof, and LNG or ENG;
a second layer arranged on the first layer and comprising ethyl cellulose, a plasticizer, and LNG or ENG; and
a third layer arranged on the first layer and comprising ethyl cellulose and a plasticizer.

15. The bioerodible film according to claim 1, wherein the film comprises a plurality of layers, wherein the layers comprise:
a first layer comprising hydroxypropyl methylcellulose, hydroxyethyl cellulose, hydroxypropyl cellulose, an acrylate-containing copolymer, polyethylene glycol, propylene glycol, EFdA and/or a salt, prodrug, or derivative thereof, and LNG;
a second layer arranged on the first layer and comprising ethyl cellulose, a plasticizer, and LNG; and
a third layer arranged on the first layer and comprising ethyl cellulose, a plasticizer, and LNG.

16. The bioerodible film according to claim 1, wherein the film comprises a plurality of layers, wherein the layers comprise:
a first layer comprising hydroxypropyl methylcellulose, hydroxyethyl cellulose, hydroxypropyl cellulose, an acrylate-containing copolymer, polyethylene glycol, propylene glycol, EFdA and/or a salt, prodrug, or derivative thereof, and LNG or ENG;
a second layer arranged on the first layer and comprising poly (lactic-co-glycolic) acid; and
a third layer arranged on the first layer and comprising poly (lactic-co-glycolic) acid.

17. The bioerodible film according to claim 14, wherein the acrylate-containing copolymer comprises an ethyl acrylate, meth methacrylate, methacrylic acid ester copolymer.

18. The bioerodible film according to claim 1, wherein the film comprises a plurality of layers, wherein a first layer comprises, by total weight of the first layer:
about 7% to about 15%, hydroxypropyl methylcellulose;
about 4% to about 10%, hydroxyethyl cellulose;
about 8% to about 20%, hydroxypropyl cellulose;
about 8% to about 20%, of an acrylate-containing copolymer;
about 6% to about 20%, polyethylene glycol; and
about 4% to about 15%, propylene glycol.

19. The bioerodible film according to claim 18, wherein a second layer of the film comprises, by total weight of the second layer:
about 70% to about 80%, ethyl cellulose; and
about 15% to about 25%, glycerin.

20. The bioerodible film according to claim 19, wherein a third layer comprises, by total weight of the third layer:
about 60% to about 85%, ethyl cellulose; and
about 15% to about 25%, of a plasticizer.

21. The bioerodible film according to claim 16, wherein the second and third layer comprise about 100%, by total weight of the layer, poly(lactic-co-glycolic) acid.

22. A method of delivering a therapeutic to a patient, comprising administering the bioerodible film of claim 1 to the patient.

23. The method according to claim 22, wherein the bioerodible film is inserted into the patient's vagina.

24. The method according to claim 22, wherein the film comprises:
an antiviral composition and provides prophylactic levels of an antiviral composition for four weeks or more; and/or
a contraceptive composition and prevents ovolation and/or fertilization for four weeks or more.

25. (canceled)

26. The bioerodible film of claim 1, wherein the film is a single layer film comprising dapivirine and/or a salt, prodrug, or derivative thereof, and LNG.
